(19) United States
(12) Patent Application Publication
(10) Pub. No.: US 2011/0184529 A1
(43) Pub. Date: Jul. 28, 2011

(54) Implants Comprising Titanium and Carbonate and Methods of Producing Implants

(76) Inventors: Johan Forsgren, Uppsala (SE); Håkan Engqvist, Osthammar (SE); Ken Welch, Sigtuna (SE)

(21) Appl. No.: 13/015,692

(22) Filed: Jan. 28, 2011

(30) Foreign Application Priority Data
Jan. 28, 2010 (SE) SE-1000091-7

(51) Int. Cl. A61F 2/28; B05D 5/00; B05D 1/36; B05D 3/02

(52) U.S. Cl. 623/23.53; 427/2.24

(57) ABSTRACT

The invention relates to a substrate comprising a bioactive element and a method to obtain the substrate with the bioactive element. The plate comprising the bioactive element relies upon formation of a carbonate layer containing biologically relevant and active ions on a surface of the substrate. Via the surface modification and mineralization, features such as bone bioactivity and/or sustained ion release can be achieved. This is beneficial for the fixation and thus the long-term outcome of implanted materials.

### TECHNICAL FIELD OF THE INVENTION

The invention relates to a device to improve tissue response to implants, and improve bone growth in the vicinity of the implant, and a method to produce such a device.

### BACKGROUND

Loosening of orthopedic and dental prostheses due to poor implant fixation is one of the most common reasons for catastrophic failure of metallic implants. In order to increase the success rate of such devices and thus reduce the increasing number of revision surgeries performed annually on failed implants, the problem with insufficient fixation must be addressed.

Cyclic loading of poorly fixated prostheses can cause implant migration. Implant migration is also known to deplete the immune defenses in the peri-prosthetic region, making it sensitive for bacteria colonization. These effects from poor implant fixation can ultimately lead to catastrophic failure of the implant where revision surgery is needed to replace the implant. Revision surgeries impose considerable strain on both patients and the health care system since they in most cases are significantly more complicated and costly compared with the primary surgery. There is a high need to increase the fixation of implants to bone to avoid or reduce the incidence of the above-mentioned problems.

There are several methods proposed in the literature to induce bone-bonding properties to titanium surfaces (Kim, H M, Miyaji, F, Kokubo, T et al. Preparation of bioactive Ti and its alloys via simple chemical surface treatment. J. Biomed. Mater. Res. 32, 409-417 (1996), Uchida, M, Kim, H M, Kokubo, T et al. Structural dependence of apatite formation on titania gels in a simulated body fluid. J Biomed Mater Res 64A, 164-170 (2003), Forsgren, J, Svahn, F, Jarmar, T et al. Formation and adhesion of biomimetic hydroxyapatite deposited on titanium substrates. Acta Biomater. 3, 980-984 (2007)). The proposed methods have general limitations when used in compromised bone e.g. osteoporotic bone, or have limited bone formation rate.

In the present patent application a material system and a manufacturing method to deliver ions locally at the site of an implant is described that improve the bone healing and implant fixation.

### SUMMARY OF THE INVENTION

An object of the present invention is to overcome one or more drawbacks of the prior art. This is achieved by the implant and method as described herein.

The invention is directed to a substrate comprising a bioactive element, and a method to produce such a substrate. Onto the substrate, an implant surface is formed, that optimizes the tissue response to metallic implants and stimulates bone growth in the vicinity of the implants. By sustained release of biologically active ions, especially Ca²⁺ and Sr²⁺ that increases the activity of bone forming cells, improved implant fixation due to stimulated bone regeneration can be achieved. Sr²⁺ has also been shown to reduce the bone resorbing activity of osteoclasts, why release of this particular ion is of special interest in patients with osteoporotic and week bone. Other ions that are also interesting to deliver or use to change the surface characteristics are Mg, Ca and Zn.

The present invention is based on surface mineralization of bioactive substrates where a carbonate layer is deposited on the surface of implants via soaking in specific salt solutions. The term “bioactivity” refers to the ability of certain materials to spontaneously form apatite on the surface when the material comes in contact with body fluids due to electrostatic interaction between the surface and different ions in solution (Lu, X & Leng, Y. Theoretical analysis of calcium phosphate precipitation in simulated body fluid. Biomaterials 26, 1097-1108 (2005). This ability can also be utilized to form artificial carbonates on surfaces in vitro as shown here. The obtained carbonate mineralized implant surface also has bioactive properties, and the apatite formed on the surface after contact with body fluids, resembles the mineral phase found in bone. This apatite layer acts like a bridging between bone and implant as cells migrates to the apatite surface and integrates it with newly formed bone surrounding the implant.

This invention describes a material system and a manufacturing method based on ion substitution in an oxide film and precipitation of a bioactive carbonate film comprising biologically active ions on metal biomedical implants where:

A Na-titanate or K-titanate surface is formed on the surface of a titanium coating or titanium or titanium alloy biomedical implant by alkali treatment. Alternatively, a layer of crystalline TiO₂ may be deposited on the surface.

The modified surface of the implant is exposed to a solution containing ions that interact and accumulate on the surface. Examples of ions are Sr, Ca, Mg and Zn. The ion deposition result in an ion-substitution in the titanate or oxide surface and also act as a nucleator for precipitation of a carbonate mineral if the ions deposited on the substrate can form complex with other ions in the solution.

The mineralized biomedical implant may further be heat-treated to induce crystallinity to the surface and thus stabilize the ion substituted oxide and the precipitated film.

The result is a composite layer surface with a titanate closest to the substrate and a carbonate layer on top.

The invention also discloses composition and performance of carbonate-mineralized surfaces with the preferred ions or combinations thereof, manufactured by the method.

The present invention is based on a biomedical implant comprising a substrate wherein the substrate comprises a first layer and a second layer, and wherein pores in the first and/or the second layer comprise at least a first element, characterized in that the first layer is a titanium containing layer and/or the second layer is a carbonate.

In one embodiment of the present invention, the substrate is titanium or a titanium alloy.

In one embodiment according to the present invention, the first layer is a titanate and/or the second layer is a carbonate. The titanate is suitably selected from sodium titanate or potassium titanate.

In one embodiment of the present invention, the first layer is titanium dioxide and/or the second layer is a carbonate.

In one embodiment of the present invention, the first element is selected from Sr, Ca, Mg, Zn or combinations thereof. The atoms of the first element can be integrated in the first layer surface.

One embodiment of the present invention discloses a method of producing a biomedical implant comprising the steps of:
Exposing the substrate to an alkali solution of NaOH or KOH, or depositing a layer of TiO₂ onto a surface of the substrate;
Exposing the substrate to an Sr and C containing solution;

The substrate may be cleaned one or several time during the process. Further, the substrate may be exposed to one or several heat treatments.

In one embodiment of the present invention, the NaOH or KOH solution preferably is in a concentration of ≧0.5M, more preferably ≧1.5M, even more preferably ≧3M, even more preferably ≧4M, even more preferably ≧5M, and the solution preferably is in a concentration of ≦10M, more preferably ≦8M, even more preferably ≦6.5M, and even more preferably ≦5M.

In one embodiment of the present invention, step of exposing the substrate to an alkali solution preferably lasts for ≧12 h, more preferably ≧18 h, and even more preferably ≧24 h, and preferably ≦60 h, more preferably ≦48 h, even more preferably ≦36 h, and even more preferably ≦24 h.

In one embodiment of the present invention, the step of exposing the substrate to an Sr and C containing solution lasts ≧1 day, preferably ≧3 days, more preferably ≧7 days, and ≦14 days, more preferably ≦11 days, even more preferably ≦7 days.

In one embodiment of the present invention, the temperature during the step of exposing the substrate to an Sr and C containing solution is between 40° C. to 80° C., more preferably between 50° C. to 70° C., most preferably 60° C.

In one embodiment of the present invention, the step of exposing the substrate to a heat treatment lasts ≧1 h, more preferably ≧3 h, and preferably ≦5 h. more preferably ≦3 h.

In one embodiment of the present invention, the temperature during the heat treatment preferably is ≧100° C. more preferably ≧300° C., and preferably ≦800° C., more preferably ≦600° C.

In one embodiment of the present invention, the Sr containing solution is added in a concentration of 5-200 mM, more preferably 50-150 mM, and even more preferably a 100 mM solution.

In one embodiment of the present invention the thickness of the titanate layer is preferably in the range between 10 nm and 10 μm, more preferably in the range between 100 nm and 5μ, even more preferably between 100 nm and 2 μm.

In one embodiment of the present invention the pores of in the titanate layer have diameters in the range from 500 nm to below 10 nm.

In one embodiment of the present invention the thickness of the carbonate layer produced is preferably in the range between 10 nm and 10 μm, more preferably in the range between 100 nm and 5μ, even more preferably between 100 nm and 2 μm.

In one embodiment of the present invention the carbonate layer is formed in the pores of the titanate layer and on top of the titanate layer.

In one embodiment of the present invention the pores of in the carbonate layer have diameters in the range between 500 nm to below 10 nm.

### BRIEF DESCRIPTION OF THE DRAWINGS

The invention will be more fully understood in view of the drawings in which:

FIG. 1 is a flowchart describing the manufacturing process of the carbonate mineralized implants surfaces.

FIG. 2 is an XRD pattern of a NaOH-treated Ti substrate exposed to a 40 mM Sr-acetate solution for 4 days and a subsequent heat treatment at 600° C. The peaks in the pattern indicate a formation of SrCO₃ on the surface. The Rutile TiO₂ peak stem from the substrate.

FIG. 3 is TEM-images at different magnifications of the porous and SrCO₃ mineralized surface. The images also display the dense interface between the titanium substrate and the mineralized volume consisting of Sr-titanate on top of Ti-oxide.

FIG. 4 is SEM images of SrCO₃-mineralized titanium substrates where a) the substrate was subjected to a heat treatment before the Sr-acetate exposure and b) the substrate was subjected to a heat treatment after the Sr-acetate exposure.

FIG. 5 is SEM images of the SrCO₃-mineralized surfaces after SBF exposure where a) the substrate was subjected to a heat treatment before the Sr-acetate exposure and b) the substrate was subjected to a heat treatment after the Sr-acetate exposure.

FIG. 6 shows the cell viability and ALP activity of the MG-63 osteoblast like cells seeded on different substrates, SrCO₃ and CaCO₃ mineralized substrates as well as on the bottom of cell culture well plates (Thermanox). A pronounced increase in call activity can be seen for the cells seeded on the mineralized surfaces, especially for the SrCO₃ surface. The decrease in activity after 3 days correlates to the lower release rate of ions after a couple of days. Since the medium was exchanged in connection to each time point for measurement, the concentration of Sr²⁺ and Ca²⁺ vin the medium became lower after 3 days.

FIG. 7 show the cumulative concentration of Sr²⁺ in the cell culture medium. The ions were released from SrCO₃ mineralized surfaces. The actual ion concentration that the cells experienced was in reality lower since the medium was exchanged several times during the measurement.

### DETAILED DESCRIPTION OF THE INVENTION

The invention relates to coatings on biomedical implants. The procedure is a wet-chemical process and is suitable for deposition of carbonate mineral coatings on both open porous and non-porous substrates. For a formation of the described coatings on implants, the method described below can be used but other methods related to the invention and obvious for a person skilled in the art in view of the present description are also considered to be within the scope of the invention. FIG. 1 describes the method in a flowchart, which is explained more in detail below.

The following substrates are suitable for preparation of the carbonate mineralized surfaces: Pure titanium or titanium alloys, non-limiting examples: Ti₆Al₄V, Ti₆Al₇Nb, Ti₃₀Nb, Ti₁₃Nb₁₃Zr, Ti₁₅Mo, Ti_{35.3}Nb_{5.1}Ta_{7.1}Zr, Ti₂₉Nb₁₃Ta_{4.6}Zr, Ti₂₉Nb₁₃Ta₂Sn, Ti₂₉Nb₁₃Ta_{4.6}Sn, Ti₂₉Nb₁₃Ta₆Sn, or Ti₁₆Nb₁₃Ta₄Mo, or coatings of the mentioned materials on any type of surface. The coatings can be deposited using any depositing technique such as plasma spraying, physical or chemical vapor deposition, sol-gel and the like.

Before preparation, the surface is suitably cleaned using common cleaning methods, preferably as follows: ultrasonic cleaning in hot water with detergent for about 5 minutes, rinsing in deionized water, ultrasonic cleaning in ethanol or acetone and blow-drying in N₂ gas.

Preparation of the carbonate mineralized bioactive surfaces containing any preferable type of ion or combinations of ions, is performed by a stepwise procedure where the substrate is exposed to an alkali-solution (alternatively, a layer of crystalline TiO₂ may be deposited on the surface) prior to an exposure to a salt solution containing the ions that are supposed to take part in the mineralization.

First the substrate is immersed in a 0.5-10M NaOH or KOH aqueous solution for 1-48 h at a temperature in the interval >0 to 95° C., preferably about 5 M NaOH for 24 hours at about 60° C. This results in the formation of a titanate surface structure of below 10 micrometer thickness. Alternatively, a layer of crystalline TiO₂ may be deposited on the surface.

Subsequently, the sample is thoroughly rinsed with water and cleaned before immersed in the salt solution for 1-14 days at >0-95° C., preferably about 7 days at 60° C. This results in ion exchange in the titanate surface and the formation of a carbonate coating on the titanate surface as formed in the previous step of below 40 micrometer thickness. This carbonate layer could be formed both in the pores of the titanate surface as well as on top of the titanate surface or both in the pores and as a layer on top of the titanate surface. The second layer can fill parts or all of the pores in the first layer. As an example; for production of Sr-carbonate, a 5-200 mM aqueous solution of Sr-acetate can be used, preferably 100 mM. For a person skilled in the art, it is obvious that it is possible to exchange the acetate to other salts or for solutions containing Ca, Mg, or Zn ions with their respective salts. Carbon in any form that can form carbonates needs to be present in the solution and can either be added to the solution as a soluble salt or from a gas source.

After the immersion in the salt solution, the sample could optionally be subjected to a heat-treatment at 100-800° C. for 1-5 h. As an alternative, the heat-treatment may be performed after the alkali-treatment instead. The method described above results in a porous layer of SrCO₃ (strontianite) formed on the titanium substrate.

The produced carbonate mineralized surface becomes highly porous with pore diameters ranging from about 500 nm to below 10 nm. The porous layer can be produced with a thickness ranging from 10 nm to 10 μm and consists of a porous titanate based network mineralized with a carbonate compound. The carbonate mineral can be amorphous to nanocrystalline depending on the fabrication route, where the amorphous phase is the most soluble one. Between the titanium substrate and the carbonate mineralized surface layer, a titanite interface is formed. Depending on the fabrication route, different types of ions can be incorporated in this titanate interface to form either Na-titanate, Sr-titanite, Ca-titanate or the like. A heat-treatment of the sample can be employed to stabilize the surface layer and induce crystallinity to the carbonate layer in order to reduce the solubility of the mineral. The heat treatment can also alter the chemistry of the titanate interface as it allows enhanced ion exchange in the titanate.

The carbonate mineralized surface acts as a reservoir of ions for local delivery. Normally the release of Sr, Ca, Mg, or Zn or combinations thereof continues for more than 24 hours and up to several months.

The surface modifications described in this invention can be used on implants to be in contact with tissue, preferably bone tissue, such as dental or orthopedic implants.

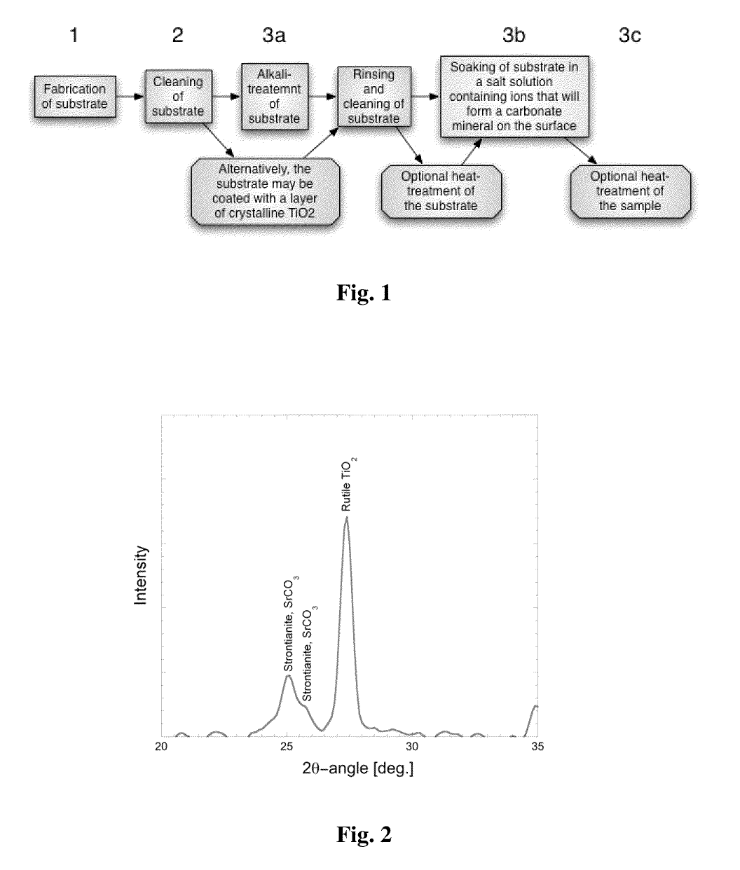

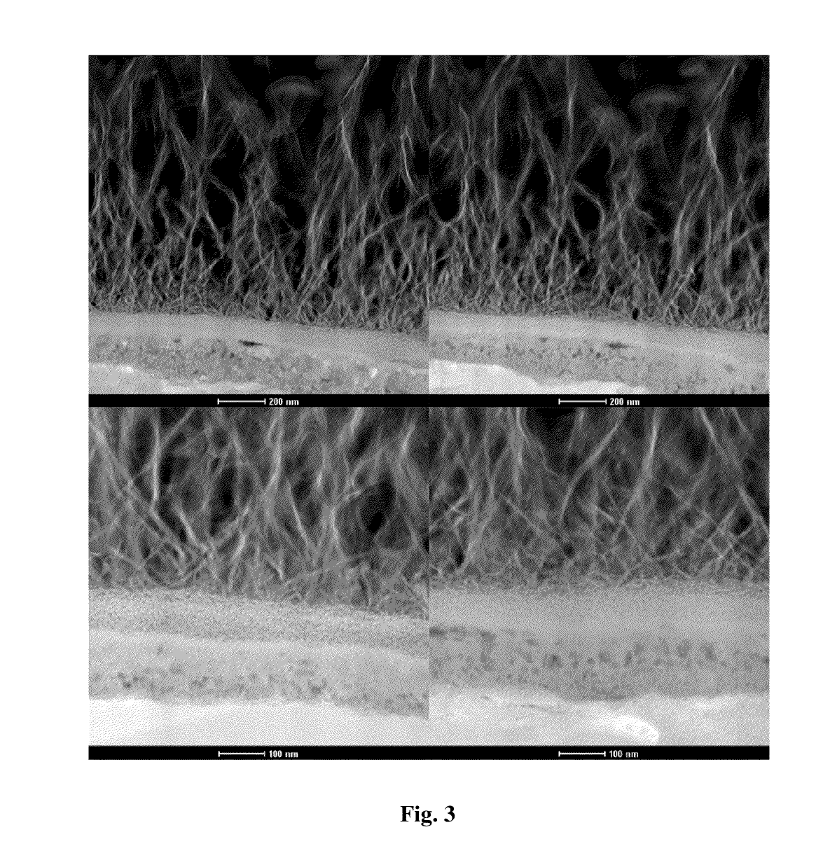

Example 1. A titanium substrate of commercially pure (purity grade 2) was immersed in an aqueous solution of 5M NaOH at 60° C. for 24 h and then placed in a beaker with a 40 mM strontium acetate aqueous solution at 60° C. for additionally 4 days. The substrate was then subjected to a heat treatment at 600° C. for 2 h. The result was a formation of SrCO₃ on the substrate as confirmed by X-ray diffraction (XRD), see FIG. 2. Transmission electron microscopy analysis (see FIG. 3) combined with energy dispersive X-ray spectroscopy reviled that the thickness of the mineralized volume was about 1.5 μm. This surface layer consisted of a porous network of titanium or Sr-titanate mineralized with nanocrystalline SrCO₃. The interface between the porous volume and the Ti substrate consisted of a ca 100 nm thick and dense layer of Sr-titanate on top of Ti oxide. The morphology of the surface can be seen in FIG. 4. The width of the pores in the mineralized surface is in the range from ca 200 nm and down to under 10 nm.

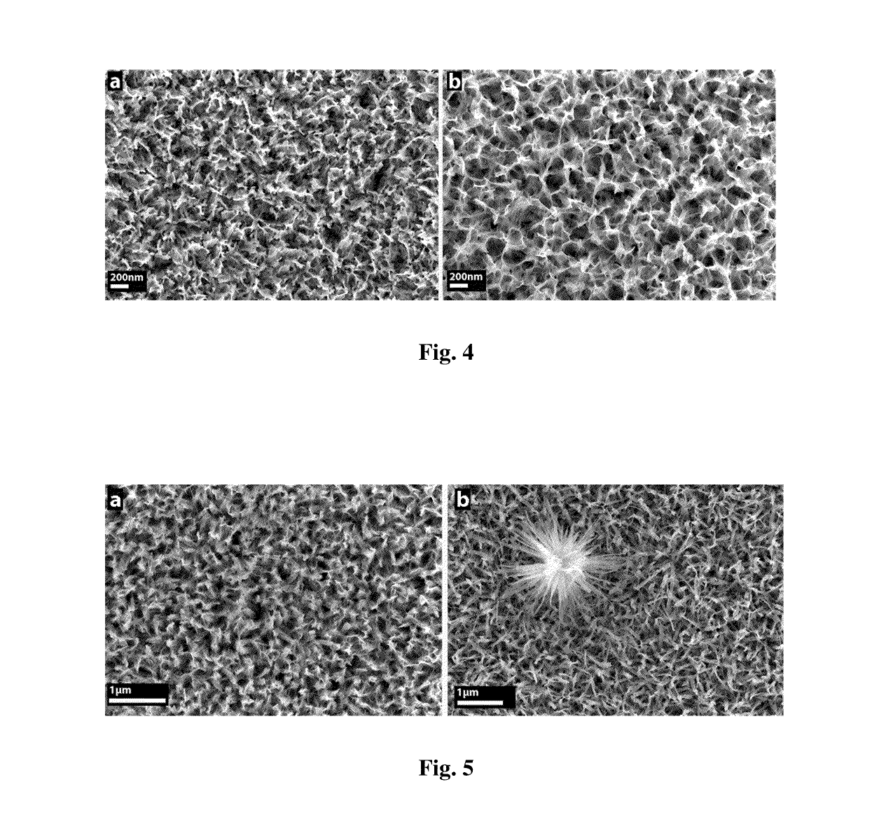

Example 2. A titanium substrate of grade 2 was immersed in an aqueous solution of 5M NaOH at 60° C. for 24 h and then subjected to a heat treatment at 600° C. for 2 h. The substrate was then placed in a beaker with a 40 mM strontium acetate aqueous solution at 60° C. for 4 days. The result was a precipitated film of SrCO₃ on the substrate (see FIG. 4). The surfaces from example 1 and 2 were tested for bone bioactivity via soaking in simulated body fluids according to the method elsewhere (Kokubo, T & Takadama, H. How useful is SBF in predicting in vivo bone bioactivity? Biomaterials 27, 2907-2915 (2006)). Both surfaces showed formation of Sr-substituted hydroxyapatite coatings on the surfaces (see FIG. 5.). X-ray photoelectron spectroscopy (XPS) analysis proved the presence and release of different ions in the surfaces before and after the SBF exposure. This proves the surfaces to be both bioactive and having an inherent ion release mechanism.

Example 3. Titanium substrates of grade 2 were immersed in an aqueous solution of 5M NaOH at 60° C. for 24 h and before placed in beakers containing aqueous solutions of either

### 100 mM Sr-acetate

### 100 mM Ca-acetate or

### 50 mM Sr-acetate and 50 mM Ca-acetate

at 60° C. for additionally 4 days. The samples were then subjected to a heat treatment at 600° C. for 2 h. The result was precipitated films of SrCO₃, CaCO₃ and a combination of SrCO₃ and CaCO₃. The formation of the different surface minerals was confirmed with XRD and XPS, see atomic concentration table obtained from the XPS analysis in Table 1.

TABLE 1
Concentration table (At %) of elements present in
the mineralized surfaces produced in Example 3.

| Element | SrCO₃ | CaCO₃ | Sr/Ca—CO₃ |
|---|---|---|---|
| O | 54.4 | 53.7 | 53.0 |
| Ti | 22.8 | 22.8 | 22.3 |
| C | 12.2 | 13.5 | 12.8 |
| Ca | 0.6 | 8.5 | 5.6 |
| Sr | 8.6 | 0.0 | 4.5 |
| Na | 1.4 | 1.5 | 1.8 |

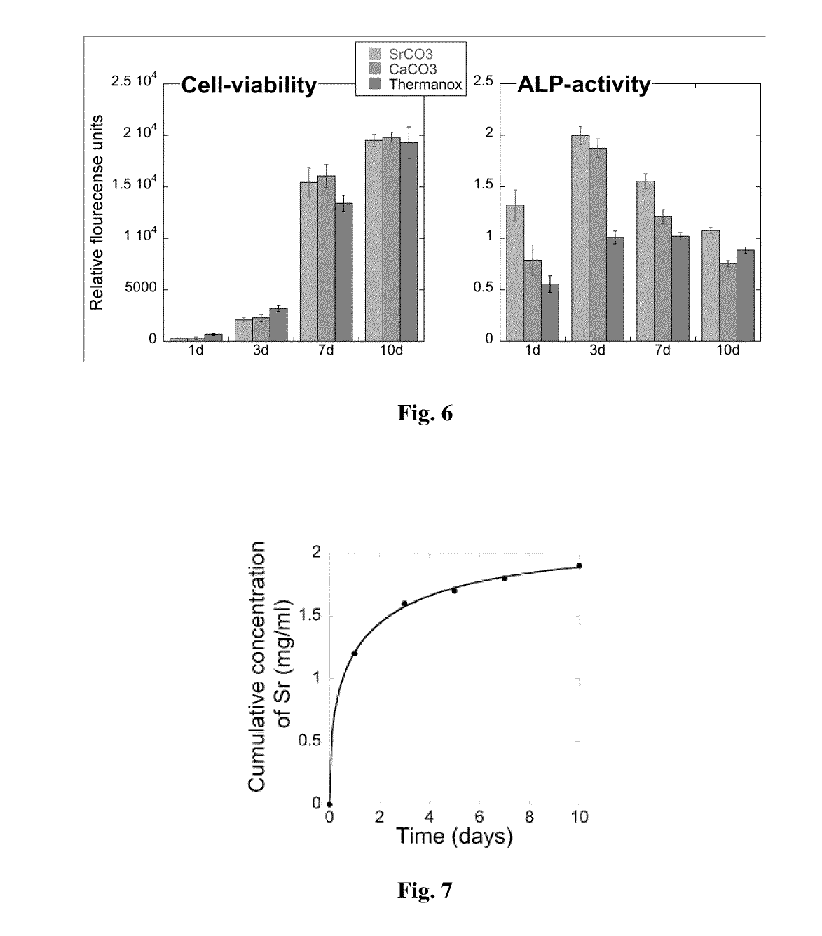

Example 4. SrCO₃ and CaCO₃ mineralized surfaces fabricated as in Example 3 were evaluated in an in vitro cell study with MG-63 human osteoblast-like cells. The cell proliferation and cell activity (ALP-expression) was measured during a 10 day study and the cell response was well correlated with the release of ions (measured with inductively coupled plasma) where the release of Sr had the highest influence on the cells, see FIGS. 6 and 7. Since the cell culture medium was exchanged in connection with each measurement of the cell activity, the concentration of ions in the medium decreased after 3 days, according to the decrease in release rate after a couple of days seen in FIG. 6. The cell activity was significantly increased on the mineralized surfaces compared to the cells seeded on the bottom of well plates (Thermanox). No signs of cytotoxicity were observed for any of the surfaces.

What is claimed is:

1. A biomedical implant, comprising a substrate wherein the substrate comprises a first layer and a second layer, and wherein pores in the first and/or the second layer comprise at least a first element,
characterized in that
the first layer is a titanium containing layer and/or the second layer is a carbonate.

2. Implant according to claim 1, wherein the substrate plate is titanium or a titanium alloy.

3. Implant according to claim 1, wherein the first layer is a titanate and/or the second layer is a carbonate.

4. Implant according to claim 3, wherein the titanate is selected from sodium titanate or potassium titanate.

5. Implant according to claim 1, wherein the first layer is titanium dioxide and/or the second layer is a carbonate.

6. Implant according to claim 1, wherein the first element is selected from Sr, Ca, Mg, Zn or combinations thereof.

7. Implant according to claim 6, wherein atoms of the first element are integrated in the first layer.

8. A method of producing a biomedical implant according to claim 1, comprising the steps of:
(a) exposing the substrate to an alkali solution of NaOH or KOH, or depositing a layer of TiO₂ onto a surface of the substrate; and
(b) exposing the substrate to an Sr and C containing solution.

9. Method according to claim 8, further comprising the step of cleaning the substrate.

10. Method according to claim 8, further comprising the step of exposing the substrate to a heat treatment.

11. Method according to claim 8, wherein the NaOH or KOH solution preferably is in a concentration of ≧0.5M, more preferably ≧1.5M, even more preferably ≧3M, even more preferably ≧4M, even more preferably ≧5M, and the solution preferably is in a concentration of ≦10M, more preferably ≦8M, even more preferably ≦6.5M, and even more preferably ≦5M.

12. Method according to claim 8, wherein the step of exposing the substrate to an alkali solution preferably lasts for ≧12 h, more preferably ≧18 h, and even more preferably ≧24 h, and preferably ≦60 h, more preferably ≦48 h, even more preferably ≦36 h, and even more preferably ≦24 h.

13. Method according to claim 8, wherein the step of exposing the substrate to an Sr and C containing solution lasts ≧1 day, preferably ≧3 days, more preferably ≧7 days, and ≦14 days, more preferably ≦11 days, even more preferably ≦7 days.

14. Method according to claim 8, wherein the temperature during the step of exposing the substrate to an Sr and C containing solution is between 40° C. to 80° C., more preferably between 50° C. to 70° C., most preferably 60° C.

15. Method according to claim 8, wherein the step of exposing the substrate to a heat treatment lasts ≧1 h, more preferably ≧3 h, and preferably ≦5 h, more preferably ≦3 h.

16. Method according to claim 11, wherein the temperature during the heat treatment preferably is ≧100° C. more preferably ≧300° C., and preferably ≦800° C., more preferably ≦600° C.

17. Method according to claim 8, wherein the Sr containing solution is added in a concentration of 5-200 mM, more preferably 50-150 mM, and even more preferably a 100 mM solution.
